(19) United States
(12) Patent Application Publication
(10) Pub. No.: US 2015/0353479 A1
(43) Pub. Date: Dec. 10, 2015

(54) CROSS METATHESIS PROCESS

(71) Applicant: ARKEMA FRANCE, Colombes (FR)

(72) Inventors: Jean-Luc DUBOIS, Millery (FR); Jean-Luc COUTURIER, Lyon (FR)

(73) Assignee: ARKEMA FRANCE, Colombes (FR)

(21) Appl. No.: 14/654,646

(22) PCT Filed: Jan. 7, 2014
(86) PCT No.: PCT/FR2014/050011

§ 371 Date: Jun. 22, 2015

(30) Foreign Application Priority Data
Jan. 7, 2013 (FR) 1350102

(51) Int. Cl. C07C 253/30

(52) U.S. Cl. CPC C07C 253/30

(57) ABSTRACT

A process for the synthesis of an unsaturated product by cross metathesis between a first unsaturated compound having at least 8 carbon atoms and a second unsaturated compound having less than 8 carbon atoms, including: feeding a reactor with the first unsaturated compound, the second unsaturated compound and a metathesis catalyst; withdrawing an output stream, at the output of the reactor; separating the output stream, making it possible to recover at least: on the one hand, the unsaturated product and, on the other hand, the first unsaturated compound and the second unsaturated compound; recycling the first unsaturated compound and the second unsaturated compound to the reactor; in which the first unsaturated compound is capable of producing an unsaturated coproduct, including at least 14 carbon atoms, by homometathesis; and in which the flow rates for feeding the reactor with first unsaturated compound and with second unsaturated compound are adjusted.

### FIELD OF THE INVENTION

The present invention relates to a cross metathesis process for the production of an unsaturated product such as an unsaturated nitrile-ester or nitrile-acid.

### TECHNICAL BACKGROUND

The polyamide industry uses an entire range of monomers formed from diamines and from diacids, from lactams, and especially from ω-amino acids. These monomers are defined by the length of the methylene chain (—CH₂)ₙ separating two amide functions —CO—NH—. These monomers are conventionally manufactured via the chemical synthesis route using, as raw materials, C₂ to C₄ olefins, cycloalkanes or benzene, which are hydrocarbons derived from fossil sources. For example, C₂ olefins are used to manufacture the C₉ amino acid used in nonanoic acid; C₄ olefins are used to manufacture hexamethylenediamine; laurolactam and caprolactam are manufactured from cycloalkanes; adipic acid, Nylon 6 and Nylon 6,6 are manufactured from benzene.

Current developments in environmental matters are leading, in the fields of energy and chemistry, to the exploitation of natural raw materials originating from a renewable source being favored. It is the reason why certain studies have been undertaken in order to industrially develop processes using fatty acids/esters as raw material for manufacturing these monomers.

Document FR 2912741 thus describes a process for the synthesis of an entire range of amino acids/esters from a natural long-chain fatty acid/ester, by subjecting the latter to a catalytic cross metathesis reaction with an unsaturated compound comprising a nitrile function, followed by hydrogenation.

Document FR 2938533 describes a process for the synthesis of ω-aminoalkanoic acids or esters thereof from natural unsaturated long-chain fatty acids, passing through an intermediate compound of ω-unsaturated nitrile type, one of the variants of which implements, in the final phase, a cross metathesis of the ω-unsaturated nitrile with a compound of acrylate type.

Document FR 2941694 describes a variant of the above process, in which the intermediate compound is of the unsaturated dinitrile type.

These processes result, at the end of a step of hydrogenation of the nitrile function and of the double bond, in the manufacture of amino acids.

Finally, the object of document FR 2959742 is to improve the performance levels of processes which successively implement a cross metathesis and a hydrogenation.

In these processes, the cross metathesis reactions, generally carried out between an omega-unsaturated fatty nitrile and an acrylate, or between an omega-unsaturated fatty ester and acrylonitrile, result not only in the desired product which is a nitrile-ester, but also in products resulting from a homometathesis reaction of fatty substances, such as respectively dinitriles and diesters. By increasing the amounts of catalyst used, the reaction times and/or the ratios between the reagents, it is possible to convert these coproducts resulting from homometathesis into a nitrile-ester, but these solutions prove to be expensive and not very productive.

Furthermore, the products of the homometathesis reactions (diesters or dinitriles) are heavy, long-chain products which have limited applications, which are often unrelated to the desired industrial applications for the nitrile-esters.

There is therefore a real need to develop a process for the synthesis of an unsaturated fatty compound by cross metathesis (and in particular for the synthesis of a nitrile-ester/acid) in which the amount of coproducts resulting from the homometathesis reactions is reduced.

### SUMMARY OF THE INVENTION

The invention relates firstly to a process for the synthesis of an unsaturated product by cross metathesis between a first unsaturated compound comprising at least 8 carbon atoms and a second unsaturated compound comprising less than 8 carbon atoms, comprising:
feeding a reactor with the first unsaturated compound, the second unsaturated compound and a metathesis catalyst;
withdrawing an output stream, at the output of the reactor;
separating the output stream, making it possible to recover at least: on the one hand, the unsaturated product and, on the other hand, the first unsaturated compound and the second unsaturated compound;
recycling the first unsaturated compound and the second unsaturated compound to the reactor;

in which the first unsaturated compound is capable of producing an unsaturated coproduct comprising at least 14 carbon atoms (preferably at least 16 and even at least 18 carbon atoms), by homometathesis; and

in which the flow rates for feeding the reactor with first unsaturated compound and with second unsaturated compound are adjusted such that the molar ratio of the net amount of unsaturated coproduct produced in the reactor to the net amount of first unsaturated compound converted in the reactor is kept below a predetermined threshold.

According to one embodiment, the predetermined threshold is 20%, or 15%, or 10%, or 5%, or 2%, or 1%, or, preferably, there is essentially no net production of unsaturated coproduct in the reactor.

According to one embodiment:
the first unsaturated compound has the formula:

R₁—CH═CH—(CH₂)ₙ—R₂; (I)
the second unsaturated compound has the formula:

R₃—CH═CH—R₄; (II)
the unsaturated product has the formula:

R₄—CH═CH—(CH₂)ₙ—R₂; (III)
the unsaturated coproduct has the formula:

R₂—(CH₂)ₙ—CH═CH—(CH₂)ₙ—R₂; (IV)

R₁ representing a hydrogen atom or an alkyl or alkenyl radical comprising from 1 to 8 carbon atoms; R₂ representing COOR₅ or CN or CHO or CH₂OH or CH₂Cl or CH₂Br; R₃ and R₄ each representing a hydrogen atom or an alkyl radical comprising from 1 to 4 carbon atoms or COOR₅ or CN or CHO or CH₂OH or CH₂Cl or CH₂Br, R₃ and R₄ being identical or different and not comprising in total at least 6 carbon atoms; R₅ representing a hydrogen atom or an alkyl radical comprising from 1 to 4 carbon atoms; and n being an integer from 4 to 11.

According to one embodiment, the second unsaturated compound is an acrylate or preferably acrylonitrile, the first unsaturated compound is an unsaturated acid, an unsaturated nitrile or an unsaturated ester, preferably chosen from methyl 9-decenoate, 9-decenenitrile, 10-undecenenitrile and methyl 10-undecenoate, the unsaturated product is an unsaturated nitrile-ester, an unsaturated nitrile-acid, an unsaturated dinitrile (by reaction of acrylonitrile with a fatty nitrile) or an unsaturated diester (by reaction of an acrylate with a fatty ester), and the unsaturated coproduct is an unsaturated diester, dinitrile or diacid.

According to one embodiment, the metathesis reactions are carried out in the liquid phase, where appropriate in a solvent, and preferably result in the production of at least one unsaturated compound in gas form, more particularly preferably ethylene, in the reactor, the process comprising the continuous drawing off thereof from the reactor.

According to one embodiment, the degree of conversion of the first unsaturated compound is from 30% to 90%, preferably from 40% to 90%, preferably from 50% to 90%, preferably from 55% to 85%, more particularly preferably from 60% to 80%.

According to one embodiment, the process is a continuous process.

According to one embodiment, the unsaturated coproduct is also recovered by separation of the output stream, and recycled to the reactor, and preferably the unsaturated coproduct load remains essentially constant.

According to one embodiment, the separation of the output stream comprises:
a first separation which makes it possible to recover the second unsaturated compound and, where appropriate, the solvent;
a second separation which makes it possible to recover the first unsaturated compound; and
a third separation which makes it possible to recover, on the one hand, the unsaturated product and, on the other hand, the unsaturated coproduct.

According to one embodiment, the flow rates for feeding the reactor with first unsaturated compound and with second unsaturated compound are adjusted such that the molar concentrations of the first unsaturated compound, of the second unsaturated compound, of the unsaturated product and of the unsaturated coproduct in the reactor are kept equal, to within 20%, preferably to within 15%, or to within 10%, or to within 5%, to reference concentrations, said reference concentrations being the respective molar concentrations of the first unsaturated compound, of the second unsaturated compound, of the unsaturated product and of the unsaturated coproduct for which the function of the yield of unsaturated coproduct relative to the degree of conversion of the first unsaturated compound exhibits a maximum, in a semi-batch reference process without recycling to the reactor, the process and the reference process being carried out under the same conditions of temperature, pressure and catalyst feed flow rate.

In the present description of the invention:
the term “molar concentration” of a compound is intended to mean the ratio of the number of moles of this compound to the volume of the reaction medium;
the term “to within X %” means that the molar concentration is included in the range from −X % to +X % relative to the corresponding reference molar concentration.

According to one embodiment, the flow rates for feeding the reactor with first unsaturated compound and with second unsaturated compound are equal to the product of the instantaneous turnover number of the catalyst multiplied by the catalyst feed flow rate.

According to one embodiment, the process is carried out in a variable volume reactor.

According to one embodiment, the process comprises, repeatedly, the following successive phases:
(1) feeding of the reactor with the catalyst, the first unsaturated compound and the second unsaturated compound and reaction between the first unsaturated compound and the second unsaturated compound for a predetermined duration;
(2) partial emptying of the reactor making it possible to withdraw the output stream;
(3) separation of the output stream making it possible to recover at least: on the one hand, the unsaturated product and, on the other hand, the first unsaturated compound and the second unsaturated compound;
(4) recycling of the first unsaturated compound and of the second unsaturated compound both resulting from the output stream to the reactor, then return to phase (1).

According to one embodiment, the process comprises, in phase (3), the recovery of the unsaturated coproduct, said coproduct not being recycled to the reactor in phase (4).

According to one embodiment, the duration of phase (1), the feed flow rates during phase (1) and the volume emptied in phase (2) are adjusted such that the molar concentrations of the first unsaturated compound, of the second unsaturated compound, of the unsaturated product and of the unsaturated coproduct in the reactor are kept equal, to within 100%, preferably to within 80% and more particularly to within 50% or to within 25%, to reference concentrations, said reference concentrations being the molar concentrations of the first unsaturated compound, of the second unsaturated compound, of the unsaturated product and of the unsaturated coproduct for which the function of the yield of unsaturated coproduct relative to the degree of conversion of the first unsaturated compound exhibits a maximum, in a semi-batch reference process without recycling to the reactor, the process and the reference process being carried out under the same conditions of temperature, pressure and catalyst feed flow rate.

Thus, according to one embodiment, the molar concentration of unsaturated product varies in a range from −80% to +50%, preferably from −50% to +25%, relative to the reference molar concentration.

According to one embodiment, the volume emptied in phase (2) is less than or equal to 80%.

According to one embodiment, the process comprises a preliminary analysis step comprising:
carrying out the reference process;
determining the yield of unsaturated coproduct as a function of the degree of conversion of the first unsaturated compound; and
determining the reference concentrations of the first unsaturated compound, of the second unsaturated compound, of the unsaturated product and of the unsaturated coproduct.

A subject of the invention is also a process for the synthesis of an α,ω-aminoalkanoic acid or ester, comprising the synthesis of an unsaturated product according to the process described above, which is an unsaturated nitrile-ester or nitrile-acid, and a reaction for hydrogenation thereof.

The present invention makes it possible to overcome the drawbacks of the prior art. It provides more particularly a process for the synthesis of an unsaturated fatty compound by cross metathesis (and in particular for the synthesis of a nitrile-ester/acid) in which the amount of coproducts resulting from the homometathesis reactions is reduced and can be controlled.

The invention is based on an analysis of the yield, from the reaction, of unwanted coproduct, as a function of the degree of conversion of the heaviest starting unsaturated compound (in particular unsaturated ester, nitrile or fatty acid). It has been discovered, surprisingly, that, when this yield exhibits a local maximum for a certain value of the degree of conversion of the starting unsaturated compound, the synthesis no longer produces coproduct at this operating point. Consequently, when the synthesis is carried out under conditions in which this degree of conversion is obtained, the addition of reagents no longer produces any new unwanted coproduct molecule. In addition, when the synthesis is carried out under conditions in which the degree of conversion is close to this optimal degree of conversion, the addition of reagents produces only a small amount of unwanted coproduct.

The use of a reactor operating in batch or semi-batch mode does not make it possible to work at a degree of conversion fixed at a desired value or fixed in a desired range, and therefore does not make it possible to adjust the amount of coproduct generated to a predetermined value.

On the other hand, the implementation of a continuous process or of another process which provides for withdrawal of reaction products, with recycling of the reagents, makes it possible, by adjusting the flow rates for feeding the reagents and the catalyst, to maintain the reactor at the optimal operating point or in the region thereof.

The implementation of a continuous process makes it possible to achieve an instantaneous selectivity close to 100% in terms of desired product, and close to 0% in terms of unwanted coproduct, by carrying out the reaction under the optimal operating conditions. The instantaneous selectivity corresponds to the molar ratio of the net amount of first compound converted into product under consideration to the net amount of first unsaturated compound which is converted (consumed) overall, taking into account all the metathesis reactions which take place in the reactor.

It is also possible to adjust the selectivities to different levels, if it is desired to produce a (predetermined) certain amount of coproduct.

The implementation of a process alternating between operating in batch mode (feeding of the reactor without withdrawal) and emptying of the reactor (with recycling) also makes it possible to achieve a selectivity in terms of desired product which is relatively high (but less than 100%, for example it may be from 70% to 95%, or from 75% to 90%, or from 80% to 85%), while at the same time saving on the amount of catalyst which is consumed.

The degree of conversion obtained with the process according to the invention is also high; however, it is not at the maximum, in order to avoid too great a consumption of catalyst (the latter deactivating very rapidly).

### BRIEF DESCRIPTION OF THE FIGURES

FIG. 1 represents, diagrammatically, a facility suitable for implementing the process of the invention, in continuous mode.

FIG. 2 represents the yield of nitrile-ester (⋄) and of diester (□) (along the y-axis, in %) as a function of the degree of conversion of an unsaturated fatty ester (along the x-axis, in %), in the context of example 1 below.

FIG. 3 represents the yield of nitrile-ester (⋄) and of diester (□) (along the y-axis, in %) as a function of the degree of conversion of an unsaturated fatty ester (along the x-axis, in %), in the context of example 3 below.

FIG. 4 represents the yield of nitrile-ester and of diester (along the y-axis, in %) as a function of the degree of conversion of an unsaturated fatty ester (along the x-axis, in %), in the context of example 6 below (simulation).

### DESCRIPTION OF EMBODIMENTS OF THE INVENTION

The invention is now described in greater detail and in a nonlimiting manner in the description which follows.

### Metathesis Reaction

The invention is based on a metathesis reaction between an unsaturated fatty compound comprising at least 8 carbon atoms, called first unsaturated compound, and a functional or non-functional olefin comprising less than 8 carbon atoms, called second unsaturated compound.

The first unsaturated compound has the formula (I) R₁—CH═CH—(CH₂)ₙ—R₂ and the second unsaturated compound has the formula (II) R₃—CH═CH—R₄ with:
R₁═H or alkyl or alkenyl radical comprising 1 to 8 carbon atoms;
R₂═COOR₅, CN, CHO, CH₂OH, CH₂Cl or CH₂Br;
R₃ and R₄═H, alkyl radical having from 1 to 4 carbon atoms, COOR₅, CN, CHO, CH₂OH, CH₂Cl or CH₂Br, R₃ and R₄ being identical or different and R₃+R₄ do not comprise more than 6 carbon atoms;
R₅═H or alkyl radical comprising 1 to 4 carbon atoms;
n is an integer from 4 to 11.

The reactions involved are:
cross metathesis between the compounds (I) and (II) giving the desired “unsaturated product” of formula (III) R₄—CH═CH—(CH₂)ₙ—R₂, and the compound of formula (V) R₁—CH═CH—R₃;
another cross metathesis between the compounds (I) and (II) giving the compound of formula (VI) R₁—CH═CH—R₄, and the compound of formula (VII) R₃—CH═CH—(CH₂)ₙ—R₂;
homometathesis of the compound (I), giving the “unsaturated coproduct” which is unwanted (or wanted in a lower amount), of formula (IV) R₂—(CH₂)ₙ—CH═CH—(CH₂)ₙ—R₂, and also the compound of formula (VIII) R₁—CH═CH—R₁.

There is generally no detectable homometathesis of the compound of formula (II) when use is made of acrylonitrile or acrylates.

The above reactions are equilibrium reactions, but the equilibria can be shifted by removal of the light compounds R₁—CH═CH—R₃ and R₁—CH═CH—R₁.

Preferably, the first unsaturated compound is an acid, a nitrile or an ester and the second unsaturated compound is acrylonitrile CH₂═CH—CN or an acrylate (for example a methyl or butyl acrylate), the unsaturated product is a nitrile-acid, an ester-acid, a diester, a dinitrile or a nitrile-ester, and the unsaturated coproduct is a diacid, a dinitrile or a diester.

The first unsaturated compound may, for example, be an ester of formula CH₂═CH—(CH₂)ₙ—COOCH₃, the second unsaturated compound being acrylonitrile CH₂═CH—CN, in which case the unsaturated product is the compound NC—CH═CH—(CH₂)ₙ—COOCH₃, and the unsaturated coproduct is the diester CH₃OOC—(CH₂)ₙ—CH═CH—(CH₂)ₙ—COOCH₃. Ethylene CH₂═CH₂ is also produced both by cross metathesis and by homometathesis. It is this example which is retained for illustrating the remainder of the description below.

Besides the reactions of fatty esters with acrylonitrile, other preferred reactions are those of fatty nitriles with an acrylate, of fatty esters with an acrylate, of fatty nitriles with acrylonitrile, of fatty esters with a linear olefin, and of fatty nitriles with a linear olefin.

In one preferred embodiment, the process involves the formation of a light product which can be removed from the reaction medium by distillation, thereby making it possible to shift the equilibria toward the formation of the desired products.

Numerous catalysts exist for metathesis reactions. Mention may, for example, be made of the tungsten complexes developed by Schrock et al (J. Am. Chem. Soc. 108:2771, 1986) or Basset et al. (Angew. Chem., Ed. Engl. 31:628, 1992). More recently, catalysts termed Grubbs catalysts have emerged (see Grubbs et al., Angew. Chem., Ed. Engl. 34:2039, 1995 and Organic Letters 1:953, 1999) which are ruthenium-benzylidene complexes operating in homogeneous catalysis. Other studies have been carried out in order to produce immobilized catalysts, i.e. catalysts of which the active ingredient is that of the homogeneous catalyst, in particular ruthenium-carbene complexes immobilized on an inactive support.

The process according to the invention advantageously uses a metathesis catalyst of ruthenium-carbene type.

The ruthenium-carbene catalysts are preferably chosen from charged or uncharged catalysts of general formula:

(X₁)ₐ(X₂)_{b}Ru(carbene C) (L₁)_{c}(L₂)_{d}(L₃)ₑ

in which:
a, b, c, d and e are integers, which may be identical or different, with a and b equal to 0, 1 or 2; c, d and e equal to 0, 1, 2, 3 or 4;
X₁ and X₂, which may be identical or different, each represent a charged or uncharged and monochelating or polychelating ligand; by way of examples, mention may be made of halides, sulfate, carbonate, carboxylates, alcoholates, phenolates, amides, tosylate, hexafluorophosphate, tetrafluoroborate, bis(triflyl)amide, an alkyl, tetraphenylborate and derivatives; X₁ or X₂ can be bonded to L₁ or L₂ or to the carbene C so as to form a bidentate or chelate ligand on the ruthenium; and
L₁, L₂ and L₃, which may be identical or different, are electron-donating ligands, such as phosphine, phosphite, phosphonite, phosphinite, arsine, stilbene, an olefin or an aromatic compound, a carbonyl compound, an ether, an alcohol, an amine, a pyridine or derivative, an imine, a thioether, or a heterocyclic carbene; L₁, L₂ or L₃ can be bonded to the carbene C so as to form a bidentate or chelate ligand, or a tridentate ligand.

The carbene C is represented by the general formula: CR₁R₂ for which R₁ and R₂ are groups which may be identical or different, such as hydrogen or any other functionalized or non-functionalized hydrocarbon-based group of saturated, unsaturated, cyclic, aromatic, branched and/or linear type. By way of examples, mention may be made of ruthenium alkylidene, benzylidene, benzylidene ether or cumylene complexes, such as vinylidenes Ru═C═CHR or allenylidenes Ru═C═C═CR₁R₂ or indenylidenes.

A functional group (making it possible to improve the retention of the ruthenium complex in an ionic liquid) can be grafted onto at least one of the ligands X₁, X₂, L₁, L₂, or onto the carbene C. This functional group may be charged or uncharged, such as preferably an ester, an ether, a thiol, an acid, an alcohol, an amine, a nitrogenous heterocycle, a sulfonate, a carboxylate, a quaternary ammonium, a guanidinium, a quaternary phosphonium, a pyridinium, an imidazolium, a morpholinium or a sulfonium.

The metathesis catalyst can optionally be rendered heterogeneous on a support in order to facilitate the recovery/recycling thereof.

The cross metathesis catalysts of the process of the invention are preferably ruthenium carbenes described, for example, in Aldrichimica Acta, vol 40, no. 2, 2007, p. 45-52.

Examples of such catalysts are Grubbs catalysts, Hoveyda-Grubbs catalysts, Piers-Grubbs catalysts, and other metathesis catalysts of the same type, whether they are “1st-generation”, “2nd-generation” or “3rd-generation” catalysts.

Grubbs catalysts are based on a ruthenium atom surrounded by 5 ligands:
2 anionic ligands, such as halides;
2 electron-donating ligands, such as trialkyl phosphines, or saturated N-heterocyclic carbenes (called NHC ligands);
an alkylidene group, such as substituted or unsubstituted methylene groups ═CR₂.

These metathesis catalysts are classified into two categories, depending on the nature of their electron-donating ligands L:
those which contain two phosphine ligands (and no saturated NHC ligand), developed first, are 1 st-generation catalysts;
those which contain a saturated NHC ligand (a heterocyclic carbene) are 2nd-generation catalysts.

A type of catalyst termed “Hoveyda-Grubbs” catalyst contains, among the electron-donating ligands, a benzylidene-ether chelating ligand, and either a phosphine (1st generation) or a saturated NHC ligand (2nd generation), usually substituted with phenyls generally substituted with mesityl (Mes) groups or else with isopropyl (iPr) groups.

Another type of catalyst termed “Piers-Grubbs” catalyst forms a four-ligand cationic complex which does not require dissociation of a ligand before the reaction.

Other types of catalysts are the “Umicore”, “Zanan” and “Grela” catalysts.

Generally, the choice of the catalyst depends on the reaction under consideration.

In one embodiment, the catalyst is free of phosphine.

Preferred catalysts are the catalysts which follow:
(1) The catalyst denoted “Hoveyda-Grubbs 2”, having the following formula:

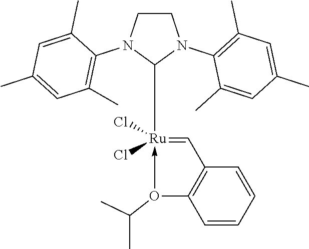

(2) The catalyst denoted “M51”, having the following formula:

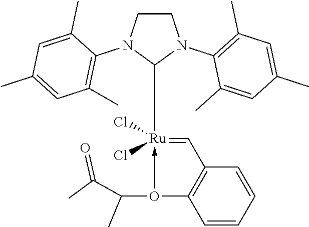

(3) The catalyst denoted “M71-SIPr”, having the following formula:

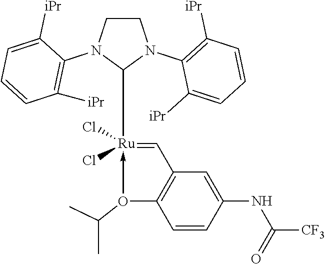

(4) The catalyst denoted “M71-SIMes”, having the following formula:

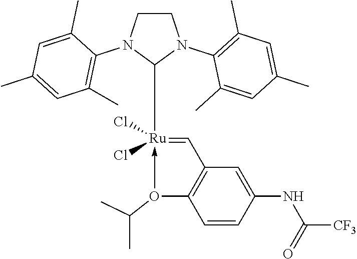

(5) The catalyst denoted “M72-SIPr”, having the following formula:

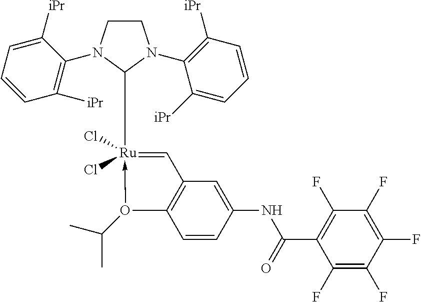

(6) The catalyst denoted “M73-SIPr”, having the following formula:

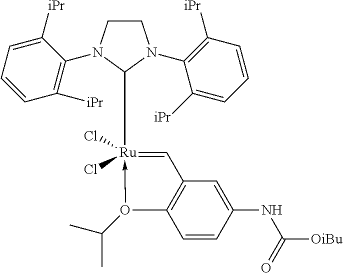

(7) The catalyst denoted “M74-SIPr”, having the following formula:

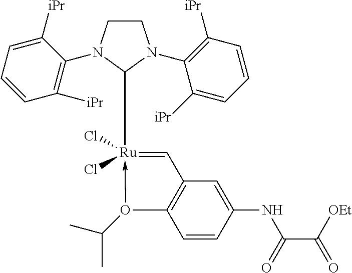

(8) The catalyst denoted “Nitro-Grela-SIMes”, having the following formula:

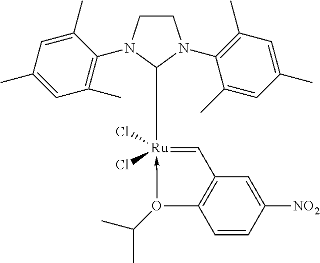

(9) The catalyst denoted “Nitro-Grela-SIPr”, having the following formula:

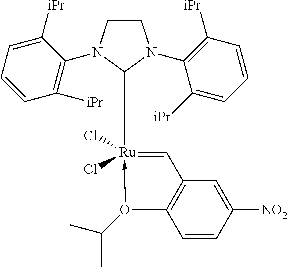

(10) The catalyst denoted “Apeiron AS2034”, having the following formula:

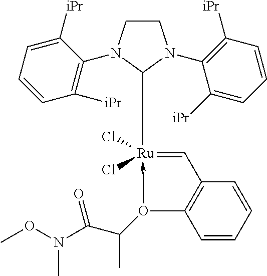

(11) The catalyst denoted “Zannan 44-0082 (Strem)”, having the following formula:

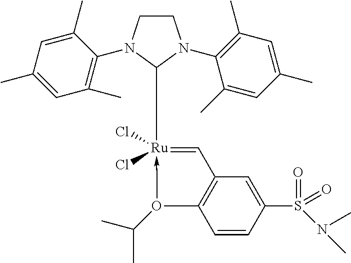

(12) The catalyst denoted “M831-SIPr”, having the following formula:

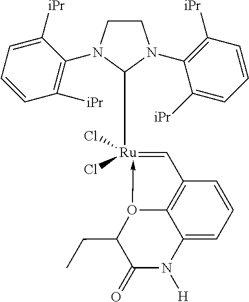

(13) The catalyst denoted “M832-SIPr”, having the following formula:

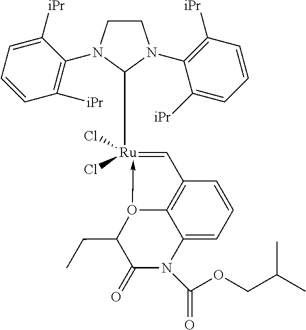

(14) The catalyst denoted “M853-SIPr”, having the following formula:

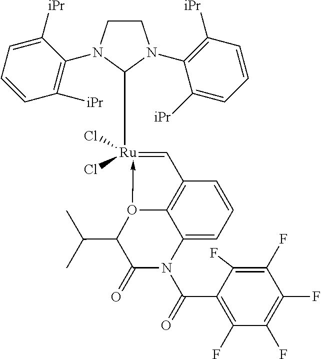

(15) The catalyst denoted “M863-SIPr”, having the following formula:

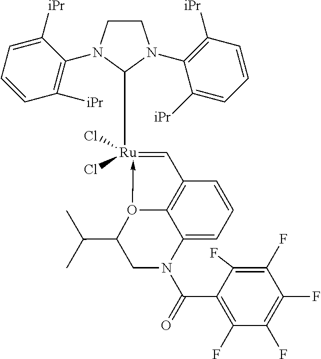

(16) The catalyst denoted “Materia C711”, having the following formula:

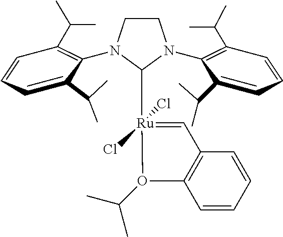

The cross metathesis reaction is optionally carried out in a solvent, in particular toluene.

### Determination of the Optimal Operating Conditions

For a given metathesis synthesis reaction, a given catalyst, and given temperature and pressure conditions, it is possible to experimentally determine optimal operating conditions, making it possible to minimize the production of the unsaturated coproduct, and therefore to achieve a selectivity of approximately 100% in terms of unsaturated product.

To do this, a synthesis process of batch type is carried out in the liquid phase. All of the first unsaturated compound (heavy compound) is introduced into the reactor, with the solvent, and also an initial amount (or all) of the second unsaturated compound (light compound). The catalyst and, where appropriate, an additional amount of the second unsaturated compound are then gradually added to the reactor in order to initiate the reaction. The continuous addition of the catalyst makes it possible to minimize the consumption thereof.

The gaseous compounds produced during the reaction (such as ethylene) are continuously removed from the reactor; thus, the cross metathesis and homometathesis reactions are not equilibrium reactions, but are totally shifted.

The gradual addition of the catalyst also makes it possible to avoid the occurrence of an excessive concentration of ethylene in the solution, which constitutes a poison for the reaction.

The composition of the reaction medium is analyzed by taking samples at regular time intervals. This makes it possible to determine at each instant, on the one hand, the degree of conversion of the first unsaturated compound (or overall degree of conversion, ODC), which corresponds to the fraction of the first unsaturated compound having reacted, and, on the other hand, the yield of the reaction (or unitary degree of conversion, UDC) in terms of unsaturated product and of unsaturated coproduct, this yield corresponding to the ratio of the number of moles of reagent actually converted into product (or into coproduct, and in this case there are 2 mol of reagent per mole of coproduct) to the number of moles of reagent introduced into the reaction medium.

Over time, the degree of conversion increases from 0% to a value which can reach more than 70%, or more than 75%, or more than 80%, or more than 85%, or even more than 90%. Thus, it is possible to establish the yield of unsaturated product as a function of the degree of conversion, and the yield of unsaturated coproduct as a function of the degree of conversion.

The inventors have noted that the yield of unsaturated product is an increasing function of the degree of conversion, while the yield of unsaturated coproduct increases and then decreases, and therefore exhibits a maximum for a certain degree of conversion (called the optimal degree of conversion), the exact value of which depends on the reaction concerned and on the implementing conditions (catalyst, temperature, pressure).

In a large number of cases, this degree of conversion is from 30% to 90%, preferably from 40% to 90%, or from 50% to 90%, or from 55% to 85%, or from 60% to 80%.

Consequently, when the concentrations of the various species are such that the degree of conversion is optimal, any (marginal) additional molecule of first unsaturated compound which is converted is converted into unsaturated product, and not into unsaturated coproduct: the selectivity in terms of unsaturated product, under these conditions (termed optimal conditions, at this particular operating point), is 100%.

Conventionally, those skilled in the art always seek to minimize coproduct formation. Consequently, using the data thus collected, they will seek to avoid the conditions corresponding to the operating point described above, where the yield of coproduct is the highest. They will seek, on the contrary, to increase the conversion so as to minimize the coproduct (but as a result will increase catalyst consumption).

By going against this conventional practice, and using a process with withdrawal and recycling of the reaction medium, the present inventors have discovered that it is possible to minimize coproduct production, while operating under conditions close to those which offer a maximum yield of coproduct. Paradoxically, it is when operating under the conditions where the yield of coproduct is at a maximum according to the prior art that the performance levels are the best according to the teaching of the invention.

The aforementioned may be understood more easily by referring to the example in which the first unsaturated compound is methyl 9-decenoate (or MD), the second unsaturated compound is acrylonitrile (ACN), the unsaturated product is methyl 10-cyano-9-decenoate (NE) and the unsaturated coproduct is methyl 9-octadecenedioate (DE).

ACN is a light compound, which has a boiling point below 100° C., while MD has a boiling point above 200° C. The reaction is carried out in a solvent medium, for example toluene, at a temperature close to 110° C. The catalyst is added continuously, over the course of 2 or 3 hours for example, and approximately half of the ACN is added before the beginning of the reaction and half during the reaction. The continuous addition of the catalyst is necessary since the catalyst deactivates very rapidly under the operating conditions. The gradual addition of ACN is made necessary by the strong inhibition of the catalyst by said ACN. There cannot therefore be a high ACN content from the beginning of the reaction.

The desired cross metathesis reaction is the reaction: ACN+MD→NE+ethylene. The ethylene produced is rapidly removed in the gas phase by entrainment with the solvent which is at its boiling point. The solvent is condensed and returned to the reactor. Owing to the continuous removal of the ethylene, the reaction is not considered to be an equilibrium reaction.

The homometathesis reaction is the reaction: MD+MD→DE+ethylene. It is also not an equilibrium reaction for the same reasons.

The final reaction which takes place is that between the ACN and the homometathesis product: ACN+DE⇄MD+NE, this reaction giving again the initial reagent (MD) and the desired product (NE). This reaction is an equilibrium reaction, the desired reaction product reacting with the initial reagent to give the homometathesis product. This reverse reaction is especially present at high conversion, when the NE concentration is high, and the conversion of the MD is already well advanced.

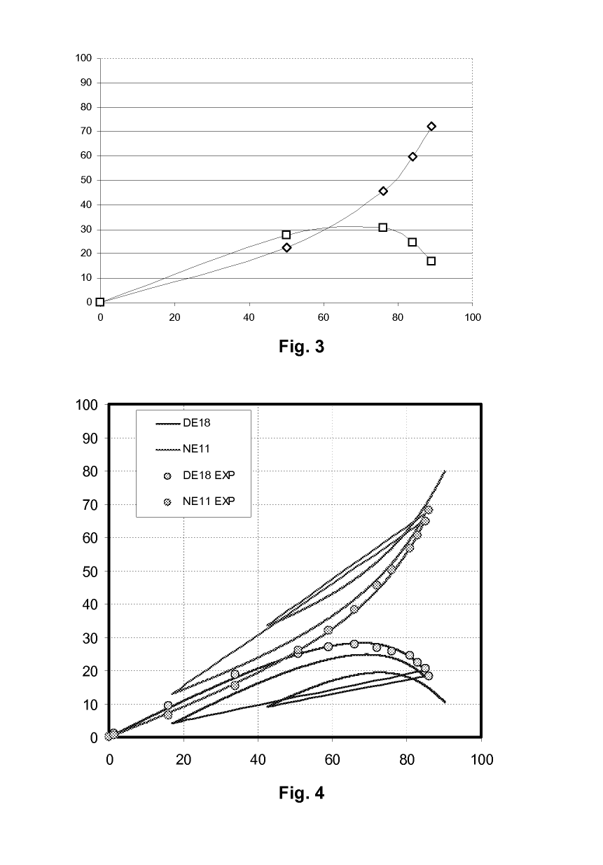

Such a reference process, making it possible to identify optimal operating conditions, is illustrated in greater detail in example 3 below, and also in FIG. 3, which shows that the unitary degree of conversion into DE is at a maximum for a certain degree of conversion of MD (approximately 70%). Under these conditions, the rate of conversion of DE is equal to its rate of formation, and DE no longer accumulates.

### Continuous Process

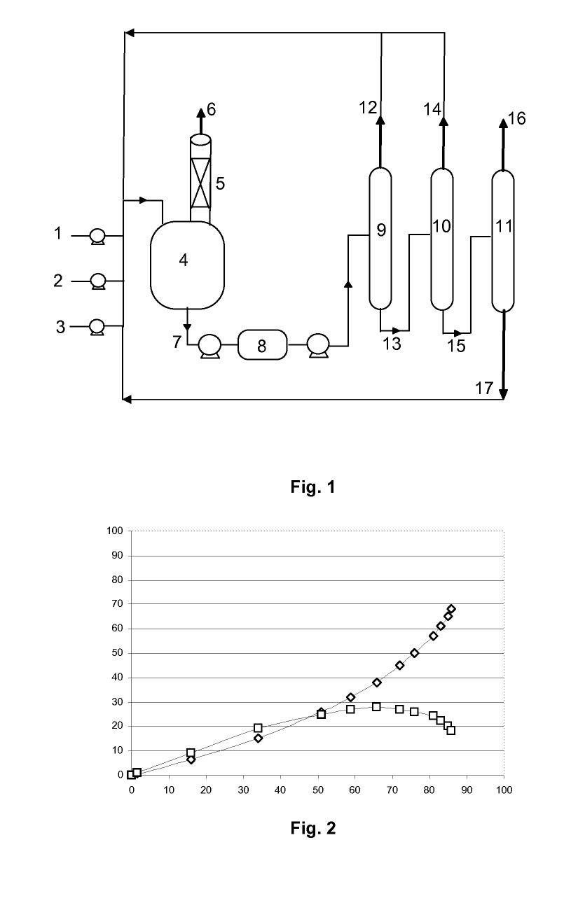

The continuous process of the invention is illustrated with reference to the facility example of FIG. 1.

The facility comprises a reactor 4, which is fed by a feed line 1 for first unsaturated compound, a feed line 2 for second unsaturated compound, and a feed line 3 for catalyst.

The reactor is equipped at the top with a gas release device 5 to which is connected a line 6 for drawing off light compounds. A line 7 for drawing off output stream is connected at the bottom of the reactor 4. Said line feeds a first distillation column 9. A storage tank 8 can be provided on the line 7 for drawing off output stream.

Connected to the top of the first distillation column 9 is a line 12 for drawing off second unsaturated compound, and connected to the bottom is a first intermediate pipe 13, which feeds a second distillation column 10.

Connected to the top of the second distillation column 10 is a line 14 for drawing off first unsaturated compound, and connected to the bottom is a second intermediate pipe 15, which feeds a third distillation column 11.

Connected to the top of the third distillation column 11 is a line 16 for drawing off unsaturated product, and connected to the bottom is a line 17 for drawing off unsaturated coproduct.

The line 12 for drawing off second unsaturated compound, the line 14 for drawing off first unsaturated compound and the line 17 for drawing off unsaturated coproduct back-feed the reactor 4.

A set of pumps make it possible to ensure circulation of the streams in the facility.

This facility makes it possible to carry out the metathesis reactions described above in the reactor 4. The lightest compounds, and in particular the ethylene, which are produced in the reactor in gas form are continuously withdrawn directly from the reactor 4, via the gas release device 5 and the line 6 for drawing off light compounds.

In addition, the liquid fraction of the reaction medium is continuously withdrawn via the line 7 for drawing off output stream. The resulting output stream is separated in the three successive distillation columns 9, 10, 11.

Going back to the above example of NE production from MD and ACN, with coproduction of DE: the acrylonitrile (ACN), and also the possible solvent of the reaction medium, are recovered via the line 12 for drawing off second unsaturated compound; the unreacted ester (MD) is recovered via the line 14 for drawing off first unsaturated compound; the desired nitrile-ester (NE) is recovered via the line 16 for drawing off unsaturated product; and, finally, the diester (DE) is recovered via the line 17 for drawing off unsaturated coproduct.

The separation of the metathesis catalyst can be carried out in various ways. By way of example, it can be carried out at the bottom of the third distillation column 11 by adsorption on an adsorbent (silica, alumina, resin, etc.) or by liquid-liquid extraction with an appropriate solvent.

All of the separated compounds are recycled to the reactor, with the exception of the desired unsaturated product, in this case NE. This recycling is supplemented by a fresh provision of reagents and of catalyst by the feed lines 1, 2, 3 connected to the reactor 4.

If care is taken to operate at concentrations of each species in the reactor such that the optimal operating conditions defined above are reached, by appropriate adjustment of the fresh species feed flow rate, there is no or virtually no diester produced and the total diester load in the facility remains constant. Alternatively, it may be chosen to withdraw (and therefore not to recycle) a part of the unsaturated coproduct, in which case the operating is adjusted so as to produce a desired flow rate of unsaturated coproduct (DE), corresponding for example to a market demand.

In what follows, an illustration of the adjustment of the flow rates enabling the reactor to operate under the optimal conditions is illustrated.

In an initial start-up phase, the process is carried out in batch mode, without drawing off output stream, in order to achieve the desired degree of conversion of MD.

Then, in steady state, the output stream is continuously drawn off, and the reactor is fed with a total flow rate equal to that of the output stream.

At the end of the start-up phase, for 100 initial mol of MD, there remain 100−X mol of MD in the reactor, where X represents the degree of conversion (as %) of the optimal operation. Making reference to FIG. 3, X is approximately 70. There are, moreover, in the reactor, X·S mol of NE, and X·(1−S)/2 mol of DE (since 2 mol of MD are consumed per mole of DE), where S represents the cumulative (or overall) selectivity of the reaction with respect to NE (desired product). X·S mol of ACN were moreover converted. Making reference to FIG. 3, for a degree of conversion of 70%, the yield of NE product is approximately 43%, which means that the cumulative selectivity S is 43/70, i.e. approximately 0.61 (or 61%).

Moreover, if Y is denoted the number of moles of ACN added since the beginning of the reaction, there are Y−X·S residual mol of ACN at the end of the start-up phase.

In steady state, the output stream keeps the same composition as that of the reaction medium at the end of the start-up phase.

All of this output stream is recycled to the reactor, with the exception of the NE, which is withdrawn. In addition, an amount X1 of fresh ACN and MD reagents is added. This amount X1 is determined by the number of moles of MD which can be converted into NE.

The following table summarizes the composition of the output stream drawn off from the reactor in steady state, and that of the mixture feeding the reactor, in steady state (ignoring the solvent, which is entirely recycled):

| | Mixture drawn off | Mixture fed |
|---|---|---|
| ACN (mol) | Y − X · S | Y − X · S + X1 |
| MD (mol) | 100 − X | 100 − X + X1 |
| NE (mol) | X · S | 0 |
| DE (mol) | X · (1 − S)/2 | X · (1 − S)/2 |

The value of X1 is calculated as a function of the flow rate of catalyst feeding the reactor. The efficiency of a catalyst is characterized by its selectivity, but also by its turnover number or TON. This is the number of moles of MD converted per mole of catalyst.

Thus, for a catalyst flow rate of Z mol, in a time t, for which X mol of MD are converted, the cumulative TON of the catalyst is X/Z. This number (TON) changes with the reaction time since the addition of catalyst is continuous. Since metathesis catalysts are complex, they are very expensive, and it is important to minimize the amounts of catalyst added. A high cumulative TON is therefore desired.

It is also possible to calculate an instantaneous TON, for a constant flow rate (d) of catalyst feeding the reactor. The cumulative TONs are calculated at any instant during the progression of the reaction (by analysis of the products and unconverted reagents of the reaction). The first derivative of this function provides the instantaneous TONs; they can also be determined experimentally, by incrementation.

In order to avoid an accumulation of reagents or products in the reactor, it is necessary to adjust the flow rate of reagents and products recycled, as a function of the catalyst flow rate.

Thus, the marginal efficiency of any mole of catalyst added corresponding to the reaction mixture in the reactor is calculated. For a conversion of X %, the instantaneous TON has a value TONᵢ(X). The number of moles of MD per unit of time which can be converted at this conversion point is therefore TONi(X)·d, which gives the flow rate of X1 moles added which can be used.

It should be noted that the aforementioned corresponds to operating under optimal conditions. However, the operating point can be modulated according to the market conditions. Thus, if the operator wishes to produce a certain amount of DE, because of the existence of a market for said DE, he chooses operating conditions to the left of the maximum on the figure, i.e. at a degree of conversion below the optimal degree of conversion, allowing him to accumulate coproduct.

Preferably, the process is carried out under conditions such that the first derivative of the function UDC(DE) relative to ODC is from −1 to +1, and preferably from 0 to 0.5 and even more preferably from 0 to 0.33, and even more preferably equal to approximately 0.

It should be noted that the above description assumes operating without losses of the compounds involved and with ideal separation of the output stream. In the event of losses and/or of imperfect separation, the fresh compound feed flow rates can be adjusted accordingly.

The degree of conversion in the case of the continuous process with recycling of the reagents and coproduct may be adjusted with respect to the reference test by adjusting the flow rate of introduction Z of the catalyst in order to compensate for a loss of activity linked to the continuous introduction of impurities in the reagents and coproducts, that are harmful to the catalyst.

Process with Partial Emptying of the Reactor

In another embodiment of the invention, the drawing off from the reactor is carried out batchwise and not continuously. Thus, at spaced-out time intervals, partial emptying of the reactor is carried out with recycling of a part of the emptied stream.

The term “variable volume operation” (or VVO) is used.

To this effect, use may, for example, be made of a variable volume reactor which has a mobile wall, such as the one described in document FR 2690926. Use may also be made of a reactor which has an overflow or a siphon.

An example of a variable volume operation is also described in section 4.6 of the article by Stankiewicz & Kuczynski, in Chemical Engineering and Processing, 34:367-377 (1995).

In this embodiment, the following successive phases are repeated:
(1) feeding of the reactor with the catalyst and the reagents (MD and ACN in the example used above);
(2) partial emptying of the reactor making it possible to withdraw an output stream;
(3) separation of the output stream making it possible to recover the desired product (NE), and also the coproduct (DE);
(4) recycling of the reagents (MD and ACN) resulting from the output stream, to the reactor, then return to phase (1), with supplementation of MD and ACN.

One advantage of this operating mode is that it avoids recycling the DE to the reactor, and creates a purge of the facility thus making it possible to reduce the concentration, in the loop, of by-products which might form.

Owing to the batchwise nature of this process, it is not carried out at a constant degree of conversion. The degree of conversion increases during phase (1), then decreases in phase (4).

The duration of phase (1) is adjusted such that the reaction is carried out in such a way as to achieve a conversion which is preferably greater than that of the maximum point (for the yield of the coproduct) determined above.

By adjusting the operating parameters (duration of phase (1), volume emptied, flow rates), it is possible to vary the degree of conversion around the optimal degree of conversion, and thus to adjust the amount of coproduct DE that is produced and also the amount of catalyst that is consumed.

This embodiment allows a lower selectivity in terms of NE than the continuous process, but a better productivity (linked to the semi-batch operation) and a lower consumption of catalyst.

According to one variant of this operating mode, phase (4) also comprises recycling of the coproduct (DE).

This variant makes it possible to avoid the accumulation of coproduct in the reactor.

An advantage of the VVO mode compared with the continuous mode is that of being able to work at a high degree of conversion of MD, higher than at the maximum point of DE, for which degree the intermediately accumulated DE is consumed, and a lower amount of diester to be recycled is thus obtained.

According to one particular embodiment of the process of the invention, the level of coproduct recycled is adjusted according to the market demand for this product.

According to another embodiment of the process of the invention, the continuous process, like the VVO process, make it possible to reach an equilibrium operating point, for which the output stream always has the same composition, thereby facilitating the downstream separation steps.

In the continuous-process embodiment, as in the VVO embodiment, the reaction is carried out at a pressure which is preferably less than 2 bar, for example equal to atmospheric pressure, or even with a partial vacuum in order to remove the light product more easily.

In the continuous-process embodiment, as in the VVO embodiment, the reaction is carried out at a temperature which is, for example, the boiling point of the solvent.

The unsaturated product obtained by virtue of the process according to the invention can undergo subsequent hydrogenation, in a manner known per se.

The work which led to this invention received financial support from the European Union in the context of Framework Program 7 (FP7/2007-2013) under project No. 241718 EUROBIOREF.

### EXAMPLES

The following examples illustrate the invention without limiting it.

### Example 1

### methyl 10-undecenoate/acrylonitrile Cross Metathesis, Reference Process

The following reaction is carried out:

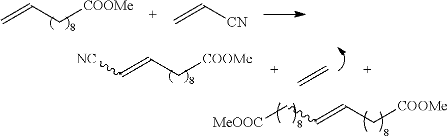

The catalyst used is provided by the company Umicore under the designation M71-SiPr. This catalyst has the following formula:

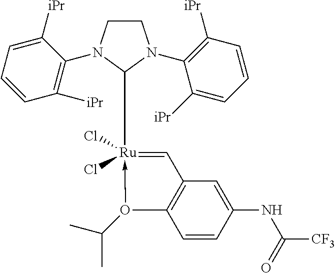

15 g of methyl 10-undecenoate (Arkema, 75.6 mmol) previously passed over an alumina column, 2 g of acrylonitrile (37.7 mmol) and 150 g of toluene dried on molecular sieve are charged to a 250 ml glass reactor equipped with a condenser and purged with nitrogen. The mixture is heated to 110° C. and 2.4 g of acrylonitrile (45.2 mmol) and 1.9 mg of M71-SiPr catalyst (2.27×10⁻⁶ mol) dissolved in 5 g of toluene are added via syringes mounted on a syringe driver, over a period of 2 h. Samples are taken every 10 minutes for analysis by gas chromatography (GC).

The conversions of the methyl undecenoate (MU) and the yields of C₁₂ unsaturated nitrile-ester (NE) and of C₂₀ unsaturated diester (DE) are reported in the graph of FIG. 2.

It is noted that the curve of DE yield as a function of the degree of conversion exhibits a maximum at approximately 65% conversion.

### Example 2

### methyl 10-undecenoate/acrylonitrile Cross Metathesis, Continuous Operation

In the light of the results of example 1, it is decided to operate, for the continuous mode experiment, under conditions which provide a degree of conversion of from 55% to 65%.

As in the case of example 1, 15 g of methyl 10-undecenoate (75.6 mmol) previously passed over an alumina column, 2 g of acrylonitrile (37.7 mmol) and 150 g of toluene dried on molecular sieve are charged to the reactor. The mixture is heated to 110° C. and 1.2 g of acrylonitrile (22.6 mmol) and 0.9 mg of M71-SiPr catalyst (1.14×10⁻⁶ mol) dissolved in 2.5 g of toluene are added with a syringe, over a period of 1 h. The reaction mixture is analyzed by GC. The composition is given in the table below.

After this start-up phase, the reaction mixture then begins to be drawn off at a flow rate of 200 ml/h via a peristaltic pump, and 200 ml of a mixture, the composition of which is given in the table below, and 0.9 mg of M71-SiPr catalyst (1.14×10⁻⁶ mol) dissolved in 2.5 g of toluene are added over the course of 1 h. After 1 h of drawing off, the composition of the mixture collected is given in the table below:

| | | Continuous | |
|---|---|---|---|
| | Start up | addition | Drawing off |
| Composition (mmol) | T = 1 h | (hourly flow rate) | T = 2 h |
| MU | 31.0 | 53.7 | 29.5 |
| Acrylonitrile | 40.1 | 55.7 | 30.3 |
| NE | 22.7 | 0 | 23.0 |
| DE | 10.9 | 10.9 | 11.4 |
| Toluene | 1655 | 1655 | ND |
| MU conversion (%) | 59 | — | 45 |
| NE selectivity (%) | 51 | — | 95 |

The continuous-mode flow rates are calculated in the same way as that which was described above:
The composition obtained at the end of the start-up is measured. This composition is drawn off at a given flow rate.
The DE which is drawn off is entirely recycled (10.9 mmol/h). The unreacted MU is entirely recycled (31.0 mmol/h) and the NE that is drawn off (22.7 mmol/h) is compensated for with fresh MU (i.e. 53.7 mmol/h in total).
The stoichiometric proportion of acrylonitrile or a slight excess relative to the MU is added (55.7 mmol/h).

The conversion after the continuous addition is less than that obtained during the start up (45% compared with 59%). This appears to be due to the fact that impurities present in the MU which are harmful to the catalyst are continuously introduced. By modifying the catalyst flow rate, it is possible to return to a conversion closer to the optimal conversion.

The results show that the amount of diester remains stable overall and that the selectivity in terms of nitrile-ester during the hour of drawing off is 95%.

### Example 3

### methyl 9-decenoate/acrylonitrile Cross Metathesis, Reference Process

The following reaction is carried out:

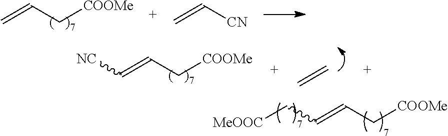

15 g of methyl 9-decenoate (81.4 mmol) prepared in accordance with example 1 of document US 2011/0113679, previously passed over an alumina column, 2.15 g of acrylonitrile (40.7 mmol) and 150 g of toluene dried on molecular sieve are charged to a 250 ml glass reactor equipped with a condenser and purged with nitrogen. The mixture is heated to 110° C. and 2.6 g of acrylonitrile (49 mmol) and 2 mg of M71-SiPr catalyst (2.44×10⁻⁶ mol) dissolved in 5 g of toluene are added via syringes mounted on a syringe driver, over a period of 2 h. Samples are taken every 30 minutes for analysis by gas chromatography. The conversions of the methyl 9-decenoate (MD) and the yields of C₁₁ unsaturated nitrile-ester (NE) and of C₁₈ unsaturated diester (DE) are reported in the graph of FIG. 3.

It is noted that the curve of DE yield as a function of the degree of conversion exhibits a maximum at approximately 70% conversion.

### Example 4

### methyl 9-decenoate/acrylonitrile Cross Metathesis, Continuous Operation

In the light of the results of example 3, it is decided to operate, for the continuous mode experiment, under conditions which provide a degree of conversion close to 70%.

As in the case of example 3, 15 g of methyl 9-decenoate (81.4 mmol) previously passed over an alumina column, 2.15 g of acrylonitrile (40.7 mmol) and 150 g of toluene dried on molecular sieve are charged to the reactor. The mixture is heated to 110° C. and 1.3 g of acrylonitrile (24.5 mmol) and 1 mg of M71-SiPr catalyst (1.22×10⁻⁶ mol) dissolved in 2.5 g of toluene are added with a syringe, over a period of 1 h. The reaction mixture is analyzed by GC. The composition is given in the table below.

The reaction mixture then begins to be drawn off at a flow rate of 200 ml/h via a peristaltic pump, and 600 ml of a mixture, the composition of which is given in the table below, and 3 mg of M71-SiPr catalyst (3.65×10⁻⁶ mol) dissolved in 5 g of toluene are added over the course of 3 h. The composition of the mixture collected hour by hour is given in the following table:

| | | Addition | | | |
|---|---|---|---|---|---|
| Composition | | (hourly flow | T = | T = | |
| (mmol) | T = 1 h | rate) | 1 h-2 h | 2 h-3 h | T = 3 h-4 h |
| MD | 29.3 | 56.4 | 28.8 | 28.8 | 29.3 |
| AN | 41.3 | 59.2 | ND | ND | ND- |
| NE | 27.1 | — | 29.0 | 27.0 | 28.4 |
| DE | 12.5 | 12.5 | 12.2 | 11.2 | 11.9 |
| Toluene | 1655 | 1655 | ND | ND | ND |
| Conv (%) | 64 | — | 49 | 49 | 48 |
| Sel (%) | 52 | — | 100 | 98 | 100 |

The amount of diester varies little during the test and the selectivity in terms of nitrile-ester is in the region of 100%.

### Example 5

### methyl 9-decenoate/acrylonitrile Cross Metathesis, Continuous Operation with Adjustment of the Catalyst Flow Rate

In this case, the process is carried out as in example 4, but 1.2 mg of M71-SIPr catalyst (1.46×10⁻⁶ mol) are added, over the course of 1 hour, after the start up.

The composition of the mixture collected is given in the following table:

| | | Addition | |
|---|---|---|---|
| Composition | | (hourly flow | |
| (mmol) | T = 1 h | rate) | T = 1 h-2 h |
| MD | 29.3 | 56.4 | 20.3 |
| AN | 41.3 | 59.2 | ND |
| NE | 27.1 | — | 35.7 |
| DE | 12.5 | 12.5 | 12.7 |
| Toluene | 1655 | 1655 | ND |
| Conv (%) | 64 | — | 64 |
| Sel (%) | 52 | — | 99 |

### Example 6

### Guide for Operation in VVO Mode

A digital simulation was carried out with operation in VVO mode, with a first emptying to ⅕th of the reactor, then a second emptying of half of the reactor, using the data of the M71 catalyst (see the illustration in FIG. 4).

It is observed that, if the same final conversion is still targeted, the DE yield decreases in the variable volume operation.

In order to obtain a stable operation, it is necessary to adjust the second final point (the term “final point” denoting the operating point at the end of phase (1)) such that it is on the straight line linking the first final point and the origin (ODC=0), in a UDC/ODC graph as represented in FIGS. 2 and 3. In this case, the conversion at the second cycle is slightly lower than that at the first, but the operation becomes more stable.

The final point is determined by selecting an operating point beyond an ideal point determined as follows:
The UDC(NE) curve as a function of time is plotted for the first cycle (which corresponds to the reference experiment).
The point of inflection of this curve (or point where the second derivative is canceled out) is determined.
At this operating point, any mole of catalyst that is added is less efficient than the previous one in terms of NE production.

The final point is therefore preferably chosen beyond this reference point. It is determined by the amount of DE that it is desired to market.

The degree of purge also determines the amount of catalyst required. If the volume purged is too small, the TON is close to the marginal TON in proximity to the final point, which is generally low. The amount of catalyst consumed in stabilized operation is therefore high.

Preferably, the return point (the term “return point” denoting the operating point at the beginning of phase (1) that follows) is chosen with respect to the point of inflection of the curve of ODC as a function of time in the reference experiment (i.e. the point where, for any mole of catalyst that is added, the number of moles of MD converted begins to drop). The return point is chosen so as to be at a conversion equivalent to or below this point of inflection. It is also such that the corresponding instantaneous TON is higher than the instantaneous TON of the final point. It is in fact desired for the difference in the cumulative TONs (final point−return point) related to the amount of catalyst used (between the return point and the final point) to be as high as possible.

In the case of the M71 catalyst, this point corresponds to approximately 50% conversion, in the case of the Hoveyda II catalyst, it corresponds to approximately 30%, for the same set of reference conditions (other than the catalyst concentration). This point therefore depends on the catalyst selected.

### Example 7

### methyl 9-decenoate/acrylonitrile Cross Metathesis, in VVO Operation

14.75 g of methyl 9-decenoate (80 mmol), 2.33 g of acrylonitrile (44 mmol) and 150 g of toluene are charged to a reactor purged with nitrogen. The mixture is heated to 110° C. and 2 mg of M71-SIPr catalyst (2.4×10⁻⁶ mol) and 2.33 g of acrylonitrile (44 mmol) are added over a period of 2 h. At the end of the addition, the reaction mixture is analyzed by GC.

The degree of conversion of the methyl 9-decenoate is 93.5%. The selectivity in terms of C₁₁ nitrile-ester is 89% and the selectivity in terms of C₁₈ diester is 11%.

During this first cycle, 75 mmol of methyl 9-decenoate were therefore converted to give 66.4 mmol of C₁₁ nitrile-ester and 4.2 mmol of C₁₈ diester.

The reactor is half emptied, then a further 7.37 g of methyl 9-decenoate (40 mmol) and 1.17 g of acrylonitrile (22 mmol) are added. At 110° C., 1 mg of M71-SIPr catalyst (1.2×10⁻⁶ mol) and 1.17 g of acrylonitrile (22 mmol) are added over a period of 2 hours.

At the end of this second cycle, the reaction mixture, analyzed by GC, comprises 5 mmol of methyl 9-decenoate, 63 mmol of C₁₁ nitrile-ester and 4 mmol of C₁₈ diester. This cycle therefore made it possible to convert 21 mmol of methyl 9-decenoate to give 11 mmol of C₁₁ nitrile-ester and 6 mmol of C₁₈ diester. The degree of conversion of the methyl 9-decenoate is 47%.

Overall, during the two cycles, 96 mmol of methyl 9-decenoate were converted, to give 77.4 mmol of C₁₁ nitrile-ester and 10.2 mol of C₁₈ diester in a reaction volume of 195 ml.

For the same conversion of the methyl 9-decenoate, this example shows the possibility of reducing the reactor size by 30% compared with a reference test in semi-batch mode (example 8) while increasing the nitrile-ester selectivity to 80.6% (compared with 65% for the reference test).

### Example 8

### methyl 9-decenoate/acrylonitrile Cross Metathesis, in Semi-Batch Mode (not in Accordance with the Invention)

22.1 g of methyl 9-decenoate (120 mmol), 3.5 g of acrylonitrile (66 mmol) and 220 g of toluene are charged to a reactor purged with nitrogen. The mixture is heated to 110° C. and 1.48 mg of M71-SIPr catalyst (1.8×10⁻⁶ mol) and 1.75 g of acrylonitrile (33 mmol) are added over a period of 1 h. At the end of the addition, the reaction mixture is analyzed by GC.

The conversion of the methyl 9-decenoate is 80%. The selectivity in terms of C₁₁ nitrile-ester is 65% and the selectivity in terms of C₁₈ diester is 35%.

This example shows that it is possible to convert 96 mmol of methyl 9-decenoate to give 62 mmol of C₁₁ nitrile-ester and 33.6 mol of C₁₈ diester in a reaction volume of 280 ml.

1. A process for the synthesis of an unsaturated product by cross metathesis between a first unsaturated compound comprising at least 8 carbon atoms and a second unsaturated compound comprising less than 8 carbon atoms, comprising:
feeding a reactor with the first unsaturated compound, the second unsaturated compound and a metathesis catalyst;
withdrawing an output stream, at the output of the reactor;
separating the output stream, making it possible to recover at least: on the one hand, the unsaturated product and, on the other hand, the first unsaturated compound and the second unsaturated compound;
recycling the first unsaturated compound and the second unsaturated compound to the reactor;
wherein the first unsaturated compound is capable of producing an unsaturated coproduct comprising at least 14 carbon atoms, by homometathesis; and
wherein the flow rates for feeding the reactor with first unsaturated compound and with second unsaturated compound are adjusted such that the molar ratio of the net amount of unsaturated coproduct produced in the reactor to the net amount of first unsaturated compound converted in the reactor is kept below a predetermined threshold, and
wherein the degree of conversion of the first unsaturated compound is from 30% to 90%.

2. The process as claimed in claim 1, wherein the predetermined threshold is 20%.

3. The process as claimed in claim 1, wherein:
the first unsaturated compound has the formula:
R₁—CH═CH—(CH₂)ₙ—R₂; (I)
the second unsaturated compound has the formula:
R₃—CH═CH—R₄; (II)
the unsaturated product has the formula:
R₄—CH═CH—(CH₂)ₙ—R₂; (III)
the unsaturated coproduct has the formula:
R₂—(CH₂)ₙ—CH═CH—(CH₂)ₙ—R₂; (IV)
R₁ representing a hydrogen atom or an alkyl or alkenyl radical comprising from 1 to 8 carbon atoms;
R₂ representing COOR₅ or CN or CHO or CH₂OH or CH₂Cl or CH₂Br;
R₃ and R₄ each representing a hydrogen atom or an alkyl radical comprising from 1 to 4 carbon atoms or COOR₅ or CN or CHO or CH₂OH or CH₂Cl or CH₂Br, R₃ and R₄ being identical or different and not comprising in total at least 6 carbon atoms;
R₅ representing a hydrogen atom or an alkyl radical comprising from 1 to 4 carbon atoms; and n being an integer from 4 to 11.

4. The process as claimed in claim 1, wherein:
the second unsaturated compound is an acrylate or acrylonitrile,
the first unsaturated compound is an acid, an unsaturated nitrile or an unsaturated ester,
the unsaturated product is an unsaturated nitrile-ester, an unsaturated nitrile-acid, an unsaturated dinitrile or an unsaturated diester, and
the unsaturated coproduct is an unsaturated diester, dinitrile or diacid.

5. The process as claimed in claim 1, wherein the metathesis reactions are carried out in the liquid phase, where appropriate in a solvent.

6. The process as claimed in claim 1, wherein the degree of conversion of the first unsaturated compound is from 40% to 90%.

7. The process as claimed in claim 1, which is a continuous process.

8. The process as claimed in claim 7, wherein the unsaturated coproduct is also recovered by separation of the output stream, and recycled to the reactor.

9. The process as claimed in claim 7, wherein the separation of the output stream comprises:
a first separation which makes it possible to recover the second unsaturated compound and, where appropriate, the solvent;
a second separation which makes it possible to recover the first unsaturated compound; and
a third separation which makes it possible to recover, on the one hand, the unsaturated product and, on the other hand, the unsaturated coproduct.

10. The process as claimed in claim 7, wherein the flow rates for feeding the reactor with first unsaturated compound and with second unsaturated compound are adjusted such that the molar concentrations of the first unsaturated compound, of the second unsaturated compound, of the unsaturated product and of the unsaturated coproduct in the reactor are kept equal, to within 20%, to reference concentrations, said reference concentrations being the respective molar concentrations of the first unsaturated compound, of the second unsaturated compound, of the unsaturated product and of the unsaturated coproduct for which the function of the yield of unsaturated coproduct relative to the degree of conversion of the first unsaturated compound exhibits a maximum, in a semi-batch reference process without recycling to the reactor, the process and the reference process being carried out under the same conditions of temperature, pressure and catalyst feed flow rate.

11. The process as claimed in claim 7, wherein the flow rates for feeding the reactor with first unsaturated compound and with second unsaturated compound are equal to the product of the instantaneous turnover number of the catalyst multiplied by the catalyst feed flow rate.

12. The process as claimed in claim 1, which is carried out in a variable volume reactor.

13. The process as claimed in claim 12, comprising, repeatedly, the following successive phases:
(1) feeding of the reactor with the catalyst, the first unsaturated compound and the second unsaturated compound and reaction between the first unsaturated compound and the second unsaturated compound for a predetermined duration;
(2) partial emptying of the reactor making it possible to withdraw the output stream;
(3) separation of the output stream making it possible to recover at least: on the one hand, the unsaturated product and, on the other hand, the first unsaturated compound and the second unsaturated compound;
(4) recycling of the first unsaturated compound and of the second unsaturated compound both resulting from the output stream to the reactor, then return to phase (1).

14. The process as claimed in claim 13, comprising, in phase (3), the recovery of the unsaturated coproduct, said coproduct not being recycled to the reactor in phase (4).

15. The process as claimed in claim 13, wherein the duration of phase (1), the feed flow rates during phase (1) and the volume emptied in phase (2) are adjusted such that the molar concentrations of the first unsaturated compound, of the second unsaturated compound, of the unsaturated product and of the unsaturated coproduct in the reactor are kept equal, to within 100%, to reference concentrations, said reference concentrations being the molar concentrations of the first unsaturated compound, of the second unsaturated compound, of the unsaturated product and of the unsaturated coproduct for which the function of the yield of unsaturated coproduct relative to the degree of conversion of the first unsaturated compound exhibits a maximum, in a semi-batch reference process without recycling to the reactor, the process and the reference process being carried out under the same conditions of temperature, pressure and catalyst feed flow rate.

16. The process as claimed in claim 10, comprising a preliminary analysis phase which comprises:
carrying out the reference process;
determining the yield of unsaturated coproduct as a function of the degree of conversion of the first unsaturated compound; and
determining the reference concentrations of the first unsaturated compound, of the second unsaturated compound, of the unsaturated product and of the unsaturated coproduct.

17. A process for the synthesis of an α,ω-aminoalkanoic acid or ester, comprising the synthesis of an unsaturated product according to the process of claim 1, which is an unsaturated nitrile-ester or nitrile-acid, and a reaction for hydrogenation thereof.
